(19) United States
(12) Patent Application Publication
(10) Pub. No.: US 2026/0097170 A1
(43) Pub. Date: Apr. 9, 2026

(54) Sealing Cap for a Syringe Tip

(71) Applicant: Becton, Dickinson and Company, Franklin Lakes, NJ (US)

(72) Inventor: Mirhac Dinc, Annecy (FR)

(21) Appl. No.: 19/115,807

(22) PCT Filed: Jul. 28, 2023
(86) PCT No.: PCT/US2023/028910

§ 371 Date: Mar. 27, 2025

(30) Foreign Application Priority Data
Sep. 28, 2022 (EP) 22198272.1

(51) Int. Cl. A61M 5/32; A61M 5/31

(52) U.S. Cl. CPC A61M 5/3202; A61M 2005/3109; A61M 2005/311

(57) ABSTRACT

A sealing cap for a syringe comprising a housing and a sealing insert received within the cavity of the housing. The housing comprises a connection portion and an insert-engagement portion, and the sealing insert comprises a tip-engagement portion and a housing-engagement portion. The housing-engagement portion of the sealing insert is received within the insert-engagement portion of the housing, and the tip-engagement portion of the sealing insert is received within the connection portion of the housing. An annular protrusion in the housing is received in an annular groove in the sealing insert. An annular locking rib extends around an outer periphery of a proximal end of the sealing insert and is positioned proximally beyond the proximal end of the housing. The engagement of the annular protrusion and the groove and between the housing and the locking rib hold the sealing insert within the housing. Also, a syringe including the sealing cap.

### CROSS-REFERENCE TO RELATED APPLICATION

The present application claims priority to European Application No. 22198272.1 entitled “Sealing Cap for a Syringe Tip” filed Sep. 28, 2022, the entire disclosure of which is incorporated by reference in its entirety.

### BACKGROUND OF THE INVENTION

### Field of the Invention

The present invention is directed to a cap for covering the tip of a syringe to keep contaminants from contacting the tip of the syringe and/or entering the fluid passageway in the tip of the syringe, thereby potentially contaminating the medicament contained in the syringe.

### Description of Related Art

For prefilled syringes, the avoidance of the contamination of the syringe tip, needle, and/or contents of the syringe is imparted by a cap provided in an interference fit with the tip and covers the tip and/or needle. A seal is formed between the cap and the tip. The seal is necessary for and contributes to the overall container closure integrity (CCI) of the syringe assembly, which is a measurement of the overall capability of the syringe assembly to keep the medicament contained in the syringe from being contaminated.

As shown in FIG. 1, prior art sealing caps 1 include a sealing insert 2 that forms the seal with the distal tip 24 of the syringe. The sealing insert 2 is surrounded by a housing 5. During placement of the sealing cap 1 on the syringe, the sealing insert 2 is pushed distally by the distal tip 24 of the syringe resulting in: (1) the upper portion 3 of the sealing insert 2 extending beyond the distal end 4 of the housing 5; (2) the engagement between a protrusion 6 in the housing 5 and a groove 7 in the sealing insert 2 intended to hold the sealing insert 2 within the housing 5 and ensure sealing contact of the sealing insert 2 with the distal tip 24 of the syringe being partially or completely disengaged; and (3) the stopper portion 8 of the sealing insert 2 intended to engage and seal the fluid passageway 26 of the distal tip 24 failing to fully engage and seal the fluid passageway 26. All of these factors negatively affect the overall sealing provided by the sealing cap 1.

There is, therefore, a need for a sealing cap that does not suffer from these deficiencies when it is placed on the distal tip of the syringe.

### SUMMARY OF THE INVENTION

The present invention is directed to a sealing cap for a syringe comprising a syringe barrel defining a reservoir for containing a medicament and a distal tip having a passageway in fluid communication with the reservoir. The sealing cap comprises a housing having an open proximal end, a distal end, and a sidewall extending from the proximal end to the distal end and defining a cavity, and a sealing insert received within the cavity of the housing. The housing comprises a connection portion and an insert-engagement portion, and the sealing insert comprises a tip-engagement portion and a housing-engagement portion. The housing-engagement portion of the sealing insert is received within the insert-engagement portion of the housing, and the tip-engagement portion of the sealing insert is received within the connection portion of the housing. An annular protrusion extends radially inwardly from the sidewall of the housing and is received in an annular groove positioned between and connecting the housing-engagement portion of the sealing insert and the tip-engagement portion of the sealing insert. An annular locking rib extends around an outer periphery of a proximal end of the sealing insert and is positioned proximally beyond the proximal end of the housing. The engagement of the annular protrusion of the housing and the groove of the sealing insert and the engagement between the proximal end of the housing and the locking rib of the sealing insert hold the sealing insert within the housing.

The tip-engagement portion of the sealing insert may include an open proximal end corresponding to the proximal end of the sealing insert, a closed distal end, and a sidewall defining a cavity extending between the proximal end of the tip-engagement portion of the sealing insert and the distal end of the tip-engagement portion of the sealing insert and adapted and dimensioned to receive the distal tip of the syringe, thereby forming a seal between an inner surface of the sidewall of the tip-engagement portion of the sealing insert and an outer surface of the distal tip. A stopper protrusion may extend from the distal end of the tip-engagement portion of the sealing insert into the cavity of the tip-engagement portion, and is disposed and dimensioned to contact and at least partially extend into the passageway of the distal tip.

The housing-engagement portion of the sealing insert extends from the tip-engagement portion of the sealing insert in a distal direction and has a distal end that corresponds to the distal end of the sealing insert.

The locking rib may be spaced apart from the proximal end of the sealing insert such that a portion of the tip-engagement portion of the sealing insert extends proximally beyond the locking rib. The locking rib may have an outer peripheral face that is angled such that an outer diameter of a distal end of the locking rib is greater than an outer diameter of a proximal end of the locking rib and/or may have a trapezoidal cross-section.

An outer diameter of the housing-engagement portion of the sealing insert may be greater than an outer diameter of the tip-engagement portion. The locking rib may have an outer diameter that is intermediate between the outer diameter of the tip-engagement portion and the outer diameter of the housing-engagement portion of the sealing insert. An outer diameter of the connection portion of the housing may be less than an outer diameter of the insert-engagement portion of the housing. A diameter of the cavity of the housing that receives the tip-engagement portion of the sealing insert may be greater than the outer diameter of the tip-engagement portion, such that space is provided between the tip-engagement portion of the sealing insert and the housing. A diameter of the cavity of the housing at the annular protrusion of the housing may be approximately equal to a diameter of the sealing insert within the groove of the sealing insert.

The insert-engagement portion of the housing may have an open proximal end, a distal end, and a sidewall defining a cavity adapted and dimensioned to receive the housing-engagement portion of the sealing insert.

The annular protrusion of the housing may extend radially inwardly around the inner surface of the sidewall of the insert-engagement portion of the housing.

The connection portion of the housing extends proximally from the insert-engagement portion of the housing and comprises an open proximal end, an open distal end, and a sidewall defining a cavity adapted to receive at least a portion of the tip-engagement portion, and threads extend around an outer peripheral surface of the sidewall of the connection portion of the housing.

The annular protrusion of the housing may be spaced a distance from the distal end of the housing that is approximately equal to or greater than a distance between the groove of the sealing insert and the distal end of the sealing insert, and/or the locking rib may be spaced a distance from the groove of the sealing insert that is approximately equal to a distance between the annular protrusion of the housing and the proximal end of the housing.

A plurality of anti-rotation ribs may extend axially in a longitudinal direction along an outer peripheral surface of the housing-engagement portion of the sealing insert and/or a plurality of anti-rotation ribs may extend axially in a longitudinal direction along an inner peripheral surface of the insert-engagement portion of the housing. The anti-rotation ribs of the housing-engagement portion of the sealing insert may be received in spaces between the anti-rotation ribs of the insert-engagement portion of the housing. A proximal end of the anti-rotation ribs of the housing-engagement portion of the sealing insert and/or a distal end of the anti-rotation ribs of the housing-engagement portion of the sealing insert may be tapered or pointed.

A material of the housing may have a higher Young's modulus, higher strength, higher hardness, and/or lower elasticity than a material of the sealing insert. The sealing insert may be made from a material selected from the group consisting of polyethylene (PE), polypropylene (PP), and an elastomeric material, and the housing may be made from a material selected from the group consisting of polycarbonate (PC), polyoxymethylene (POM), polybutylene terephthalate (PBT), polypropylene (PP), polyethylene (PE), polyvinylchloride (PVC), polystyrene, polycarbonate, acrylonitrile butadiene styrene, styrene acrylonitrile, fiber-reinforced polymer, metal, and combinations thereof.

The present invention is also directed to a syringe comprising a syringe barrel defining a reservoir for containing a medicament; a distal tip having a passageway in fluid communication with the reservoir; and a sealing cap as discussed above. The sealing cap covers a distal end of the passageway and creates a seal between the sealing insert and an outer surface of the distal tip to keep air and contaminants from entering the cavity of the sealing cap and/or the passageway.

The connection portion of the housing may be threadedly engaged with a luer collar surrounding a proximal end of the distal tip such that the distal tip is received in a cavity of the tip-engagement portion of the sealing insert. The luer collar may be a separate component that is friction fitted onto the distal tip. The luer collar may comprise a proximal end adjacent a distal face of the syringe, an open distal end, and a sidewall spaced from and at least partially surrounding the distal tip, where an inner surface of the sidewall of the luer collar includes threads that are engaged by threads on an outer surface of the connection portion of the housing. A plurality of radially inward directed mounting projections may extend from the sidewall of the luer collar at the proximal end of the luer cover and frictionally engage the distal tip.

### BRIEF DESCRIPTION OF THE DRAWINGS

FIG. 1 is a cross-sectional view of a prior art sealing cap placed on a distal tip of a syringe;

FIG. 2 is an exploded side perspective view of a sealing cap and syringe according to the invention;

FIG. 3 is a cross-sectional view of an embodiment of a sealing cap according to the invention;

FIG. 4 is a cross-sectional view of the sealing insert of the sealing cap of FIG. 3;

FIG. 5 is a cross-sectional view of the housing of the sealing cap of FIG. 3;

FIG. 6 is a side perspective view of an embodiment of a sealing cap according to the invention;

FIG. 7 is a cross-sectional view of the sealing cap of FIG. 3 placed on a distal tip of a syringe having an integral luer collar;

FIG. 8 is a cross-sectional view of the sealing cap of FIG. 3 placed on a distal tip of a syringe having a separate luer collar; and

FIG. 9 is a cross-sectional view of the luer collar of FIG. 8.

### DESCRIPTION OF THE INVENTION

As used herein, any numerical values are expressed using a period as a decimal point and a comma as a thousand separator, for example, 1,234 would be one thousand two hundred thirty four, and 1.2 would be one and two tenths. Unless otherwise expressly specified, all numbers, such as those expressing values, ranges, amounts or percentages, may be read as if prefaced by the word “about”, even if the term does not expressly appear. Any numerical range recited herein is intended to include all sub-ranges subsumed therein. For example, a range of “1 to 10” is intended to include any and all sub-ranges between and including the recited minimum value of 1 and the recited maximum value of 10, that is, all subranges beginning with a minimum value equal to or greater than 1 and ending with a maximum value equal to or less than 10, and all subranges in between, e.g., 1 to 6.3, or 5.5 to 10, or 2.7 to 6.1. Plural encompasses singular and vice versa. When ranges are given, any endpoints of those ranges and/or numbers within those ranges can be combined with the scope of the present invention. “Including”, “such as”, “for example” and like terms mean “including/such as/for example but not limited to”.

For purposes of the description hereinafter, spatial orientation terms, as used, shall relate to the referenced embodiment as it is oriented in the accompanying drawings, figures, or otherwise described in the following detailed description. However, it is to be understood that the embodiments described hereinafter may assume many alternative variations and configurations. It is also to be understood that the specific components, devices, features, and operational sequences illustrated in the accompanying drawings, figures, or otherwise described herein are simply exemplary and should not be considered as limiting.

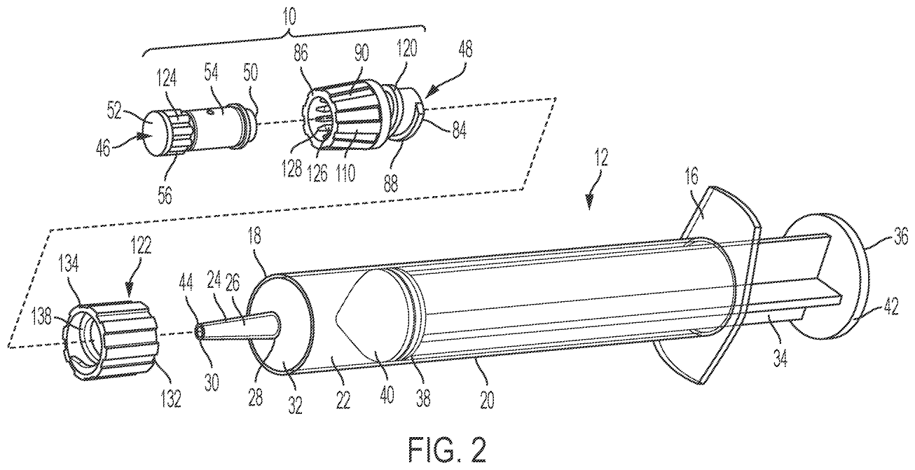

The present invention is directed to a sealing cap 10 for a syringe 12 and a syringe 12 including such a sealing cap 10 (FIG. 2).

As shown in FIG. 2, the inventive sealing cap 10 is adapted for use with a syringe 12 comprising a syringe barrel 14 having a proximal end 16, a distal end 18, and a sidewall 20 extending from the proximal end 16 to the distal end 18 and defining a reservoir 22 for containing a medicament. A distal tip 24 of the syringe 12 extends from the distal end 18 of the syringe barrel 14. A fluid passageway 26 extends through the distal tip 24 of the syringe 12 through which the medicament may be ejected from the reservoir 22.

The distal tip 24 of the syringe 12 may have an outer surface that is substantially cylindrical or the distal tip 24 of the syringe 12 may taper such that an outer diameter of the proximal end 28 of the distal tip 24 of the syringe 12 adjacent the distal end 18 of the syringe barrel 14 is larger than an outer diameter of the distal end 30 of the distal tip 24 of the syringe 12. The largest outer diameter of the distal tip 24 of the syringe 12 is smaller than an outer diameter of the syringe barrel 14, thereby forming a distal face 32 at the distal end 18 of the syringe barrel 14.

The syringe 12 may include a plunger rod 34 having a proximal end 36 and a distal end 38 with a stopper 40 extending from the distal end 38 and a pressing surface 42 extending from the proximal end 36. The stopper 40 forms a seal with the sidewall 20 of the syringe barrel 14 to contain the medicament within the reservoir 22, and the plunger rod 34 is moveable within the reservoir 22 of the syringe barrel 14 to eject the medicament from the reservoir 22 through the fluid passageway 26.

The inventive sealing cap 10 covers and seals a distal end 44 of the fluid passageway 26 and creates a seal with the distal tip 24 of the syringe 12 to keep air and contaminants from entering the fluid passageway 26 and thereby contaminating the medicament.

As shown in FIGS. 2-8, the sealing cap 10 comprises a sealing insert 46 that is received within a housing 48.

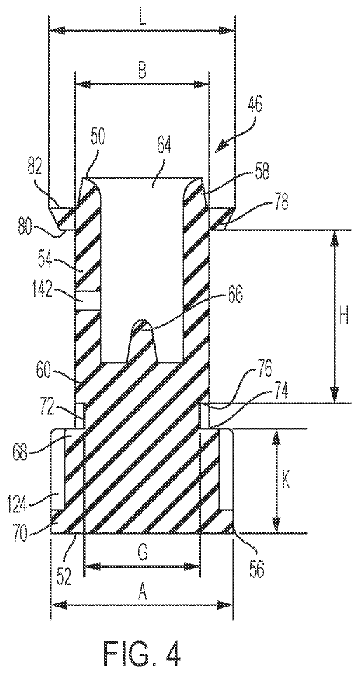

As shown in FIG. 4, the sealing insert 46 has an open proximal end 50, a closed distal end 52, a tip-engagement portion 54 at the proximal end 50, and a housing-engagement portion 56 at the distal end 52. The tip-engagement portion 54 comprises an open proximal end 58 corresponding to the proximal end 50 of the sealing insert 46, a closed distal end 60, and a sidewall 62 defining a cavity 64 extending between the proximal end 58 and the distal end 60 and adapted and dimensioned to receive the distal tip 24 of the syringe 12, thereby forming a seal between the inner surface of the sidewall 62 of the tip-engagement portion 54 of the sealing insert 46 and the outer surface of the distal tip 24 of the syringe 12. A stopper protrusion 66 extends from the distal end 60 of the tip-engagement portion 54 of the sealing insert 46 into the cavity 64 of the tip-engagement portion 54 of the sealing insert 46. The stopper protrusion 66 is disposed and dimensioned to contact and at least partially extend into the fluid passageway 26 of the distal tip 24 of the syringe 12.

The housing-engagement portion 56 extends in a distal direction and has a proximal end 68 and a distal end 70 that corresponds to the distal end 52 of the sealing insert 46.

The tip-engagement portion 54 of the sealing insert 46 is connected to the housing-engagement portion 56 of the sealing insert 46 by an annular groove 72 that extends around the periphery of the sealing insert 46. The groove 72 defines a distal face 74 at the distal end 60 of the tip-engagement portion 54 and a proximal face 76 at the proximal end 68 of the housing-engagement portion 56. The outer diameter A of the housing-engagement portion 56 of the sealing insert 46 may be greater than the outer diameter B of the tip-engagement portion 54 of the sealing insert 46.

An annular locking rib 78 extends around the outer periphery of the proximal end 58 of the tip-engagement portion 54 of the sealing insert 46. The locking rib 78 has an outer diameter L that is intermediate between the outer diameter B of the tip-engagement portion 54 of the sealing insert 46 and the outer diameter A of the housing-engagement portion 56 of the sealing insert 46. The locking rib 72 may be spaced apart from the proximal end 50 of the sealing insert 46 such that a portion of the tip-engagement portion 54 of the sealing insert 46 extends proximally beyond the locking rib 72. The locking rib 72 may have an outer peripheral face angled such that the outer diameter of the distal end 74 of the locking rib 72 is greater than the outer diameter of the proximal end 76 of the locking rib 72 and the locking rib 72 may have a trapezoidal cross-section.

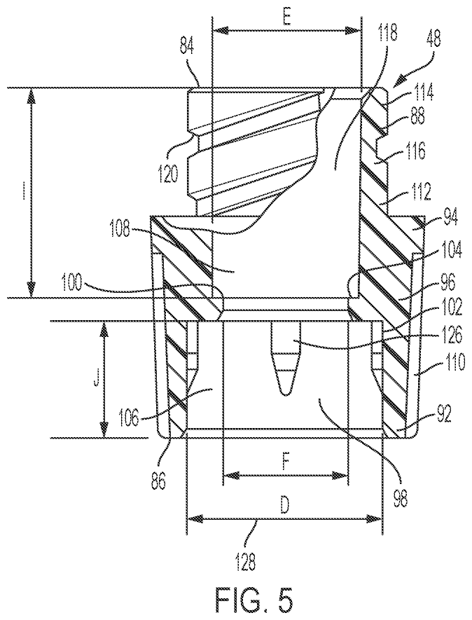

As shown in FIG. 5, the housing 48 comprises an open proximal end 84, a distal end 86, a connection portion 88 at the proximal end 84, and an insert-engagement portion 90 at the distal end 86. The housing 48 may be substantially cylindrical. The distal end 86 of the housing 48 may be open.

The insert-engagement portion 90 has an open proximal end 92, a distal end 94, and a sidewall 96 defining a cavity 98 extending between the proximal end 92 and the distal end 94 and adapted and dimensioned to receive the housing-engagement portion 56 of the sealing insert 46. The distal end 94 of the insert-engagement portion 90 may be open.

An annular protrusion 100 extends radially inwardly around the inner surface of the sidewall 96 of the insert-engagement portion 90 of the housing 48. The radial length of the distal face 102 of the protrusion 100 is larger than the radial length of the proximal face 104 of the protrusion 100, such that the diameter D of a distal portion 106 of the cavity 98 of the insert-engagement portion 90 of the housing 48 distal to the protrusion 100 is greater than the diameter E of a proximal portion 108 of the cavity 98 of the insert-engagement portion 90 of the housing 48 proximal to the protrusion 100.

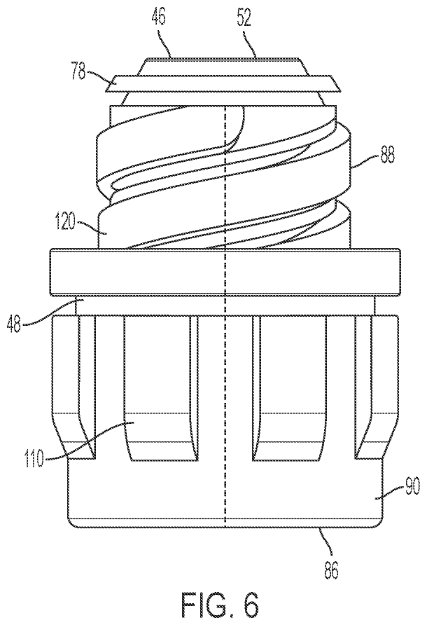

Optionally, grasping protrusions 110 may extend radially outward from an outer surface of the insert-engagement portion 90 of the housing 48 (FIG. 6). The grasping protrusions 110 are dimensioned and configured to facilitate manual gripping of the sealing cap 10 by a user during removal of the sealing cap 10 from the syringe 12. For example, the grasping protrusions 110 may be a plurality of ribs.

The connection portion 88 of the housing 48 extends proximally from the proximal end 92 of the insert-engagement portion 90 of the housing 48 and comprises an open proximal end 112, an open distal end 114, and a sidewall 116 extending from the proximal end 112 to the distal end 114 and defining a cavity 118. The diameter of the cavity 118 of the connection portion 88 of the housing 48 is the same as the diameter E of the proximal portion 108 of the cavity 98 of the insert-engagement portion 90 of the housing 48. Threads 120, which are engageable with the threads of a luer collar 122 surrounding the distal tip 24 of the syringe 12, extend around the outer peripheral surface of the sidewall 116 of the connection portion 88 of the housing 48.

The outer diameter of the connection portion 88 of the housing 48 is less than the outer diameter of the insert-engagement portion 90 of the housing 48.

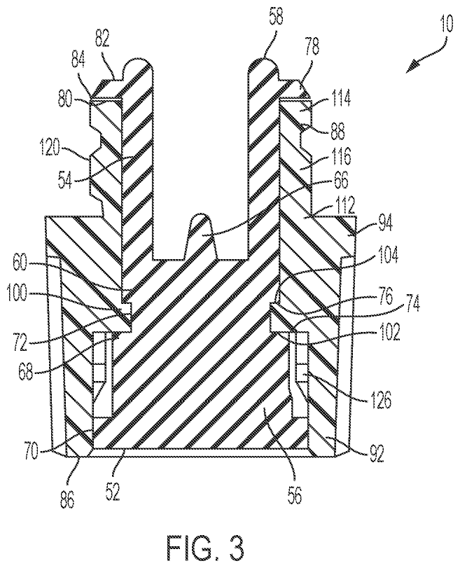

As shown in FIGS. 3, 7, and 8, the housing-engagement portion 56 of the sealing insert 46 is received within the distal portion 106 of the cavity 98 of the insert-engagement portion 90 of the housing 48, and the tip-engagement portion 54 of the sealing insert 46 is received within the proximal portion 108 of the cavity 98 of the insert-engagement portion 90 of the housing 48 and the cavity 118 of the connection portion 88 of the housing 48. The protrusion 100 of the housing 48 is received in the annular groove 72 of the sealing insert 46. The locking rib 78 of the sealing insert 46 and the proximal end 50 of the sealing insert 46 extend beyond the proximal end 84 of the housing 48 with the locking rib 78 of the sealing insert 46 engaging the proximal end 84 of the housing 48.

The diameter E of the proximal portion 108 of the cavity 98 of the insert-engagement portion 90 of the housing 48 and the cavity 118 of the connection portion 88 of the housing 48, which are equal, is greater than the outer diameter B of the tip-engagement portion 54 of the sealing insert 46, such that space is provided between the sealing insert 46 and the housing 48 to allow for expansion of the tip-engagement portion 54 of the sealing insert 46 when the sealing cap 10 is placed on the distal tip 24 of the syringe 12.

The protrusion 100 of the housing 48 is spaced a distance J from the distal end 86 of the housing 48 that is approximately equal to or greater than the distance K between the groove 72 of the sealing insert 46 and the distal end 52 of the sealing insert 46. The diameter F of the opening of the cavity 98 of the housing 48 at the protrusion 100 is approximately equal to the diameter G of the sealing insert 46 within the groove 72. The distal portion of the protrusion 100 of the housing 48 may be chamfered to facilitate deflection of the sealing insert 46 during assembly of the sealing cap 10 as will be discussed later.

The locking rib 78 of the sealing insert 46 is spaced a distance H from the groove 72 of the sealing insert 46 that is approximately equal to the distance I between the protrusion 100 of the housing 48 and the proximal end 84 of the housing 48.

The sidewall 96 of the insert-engagement portion 90 may optionally include openings (not shown) that allow for expansion of the housing-engagement portion 56 of the sealing insert 46 during assembly of the sealing cap 10, placement of the sealing cap 10 on the syringe 12, and/or removal of the sealing cap 10 from the syringe 12.

Optionally, a vent opening 142 may extend through the sidewall 62 of the tip-engagement portion 54 of the sealing insert 46 to reduce any vacuum that is created as the sealing insert 46 is removed from distal tip 24 of the syringe 12. When the sealing cap 10 is disposed on the distal tip 24 of the syringe 12, the vent opening 142 is blocked by the distal tip 24. As the sealing cap 10 is removed from the syringe 12 and the portion of the sealing insert 46 containing the vent opening 142 loses contact with the distal tip 24, the vent opening 142 allows air to enter the cavity 64 of the tip-engagement portion 54 of the sealing cap 10, minimizing any vacuum that may form as the sealing cap 10 is further removed from the syringe 12.

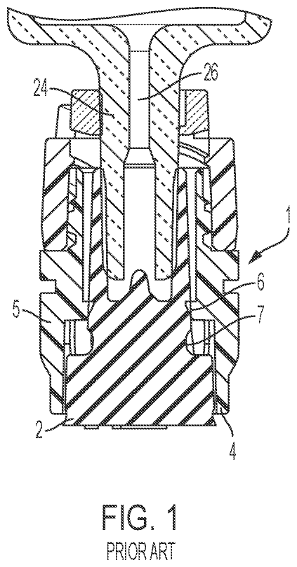

As shown in FIG. 1, prior art sealing caps 1 include a sealing insert 2 that forms the seal with the distal tip 24 of syringe. The sealing insert 2 is surrounded by a housing 5. During placement of the sealing cap 1 on the syringe: (1) the sealing insert 2 is pushed distally by the distal tip 24 of the syringe resulting in the upper portion 3 of the sealing insert 2 extending beyond the distal end 4 of the housing 5; (2) the engagement between a protrusion 6 in the housing 5 and a groove 7 in the sealing insert 2 intended to hold the sealing insert 2 within the housing 5 and ensure sealing contact of the sealing insert 2 with the distal tip 24 of the syringe being partially or completely disengaged; and (3) the stopper portion 8 of the sealing insert 7 intended to engage and seal the fluid passageway 26 of the distal tip 24 failing to fully engage and seal the fluid passageway 26. All of these factors negatively affect the overall sealing provided by the sealing cap 1.

The inventive sealing cap 10 corrects the deficiencies present in the prior art sealing cap 1. The combination of the engagement between the protrusion 100 of the housing 48 and the groove 72 of the sealing insert 46 and the engagement between the proximal end 84 of the housing 48 and the locking rib 78 of the sealing insert 46 acts to hold the sealing insert 46 within the housing 48 during placement of the sealing cap 10 on the distal tip 24 of the syringe 12 and to assure a good seal between the sealing insert 46 of the sealing cap 10 and the distal tip 24 of the syringe 12. The locking rib 78 keeps the sealing insert 46 from being pushed distally by the distal tip 24 of the syringe 12 and, thereby, keeps the insert-engagement portion 90 of the sealing insert 46 from extending beyond the distal end 86 of the housing 48, keeps the protrusion 100 of the housing 48 engaged with the groove 72 of the sealing insert 46, and keeps the stopper protrusion 66 of the sealing insert 46 fully engaged with and sealing the fluid passageway 26 of the distal tip 24 of the syringe 12.

Optionally, a plurality of anti-rotation ribs 124 may extend axially in a longitudinal direction along an outer peripheral surface of the housing-engagement portion 56 of the sealing insert 46, and/or a plurality of anti-rotation ribs 126 may extend axially in a longitudinal direction along an inner peripheral surface of the insert-engagement portion 90 of the housing 48 (FIGS. 2, 4, and 5). The anti-rotation ribs 124 of the sealing insert 46 may be received in the spaces between the anti-rotation ribs 126 of the housing 48 in order to keep the sealing insert 46 from rotating within the housing 48. The anti-rotation ribs 124 of the sealing insert 46 and/or anti-rotation ribs 126 of the housing 48 may be spaced equally around the peripheral surfaces of the housing-engagement portion 56 of the sealing insert and/or the connection portion 88 of the housing 48, respectively. For example, the anti-rotation ribs 124 and/or the anti-rotation ribs 126 may be spaced at approximately 45° from one another about the respective peripheral surface. The distal end 128 of the anti-rotation ribs 126 of the housing 48 and/or the proximal end 130 of the anti-rotation ribs 124 of the sealing insert 46 may be tapered or pointed to urge the anti-rotation ribs 124 of the sealing insert 46 into the spaces between the anti-rotation ribs 126 of the housing 48 during assembly of the sealing cap 10.

The sealing cap 10 is assembled by inserting the proximal end 50 of the sealing insert 46 into distal end 86 of the housing 48 and urging the sealing insert 46 in the proximal direction. The tip-engagement portion 54 of the sealing insert 46 engages the protrusion 100 of the housing 48 and is deflected radially inward. As the proximal movement of the sealing insert 46 is continued, the locking rib contacts the protrusion 100 of the housing 48 and the sealing insert 46 is further deflected radially inward until the locking rib 78 has passed the protrusion 100 of the housing 48. The angled outer peripheral face of the locking rib 78 assists in the deflection of the sealing insert 46 and the passage of the locking rib 78 past the protrusion 100 of the housing 48. After sufficient proximal movement of the sealing insert 46 with respect to the housing 48, the optional anti-rotation ribs 124 of the sealing insert 46 engage the anti-rotation ribs 126 of the housing 48. The pointed or tapered ends of anti-rotation ribs 124, 126 cause the sealing insert 46 to rotate slightly, such that anti-rotation ribs 124 of the sealing insert 46 align with the anti-rotation ribs 126 of the housing 48 and the sealing insert 46 can be further sealing inserted into the housing 48 with the anti-rotation ribs 124 of the sealing insert 46 positioned between the anti-rotation ribs 126 of the housing 48. Further advancement of the sealing insert 46 into the housing 48 causes the groove 72 of the sealing insert 46 to align with protrusion 100 of the housing 48 and the locking rib 78 of the sealing insert 46 to extend beyond the proximal end 84 of the housing 48. At this point, the sealing insert 46 resiliently returns toward an undeflected condition with the protrusion 100 of the housing 48 trapped in the groove 72 of the sealing insert 46 and the locking rib 78 of the sealing insert 46 engaging the proximal end 84 of the housing 48 to substantially prevent further axial movement between the sealing insert 46 and the housing 48. In this position, the interconnection of the anti-rotation ribs 124 of the sealing insert 46 and the anti-rotation ribs 126 of the housing 48 prevents or limits rotation of the sealing insert 46 relative to the housing 48.

As shown in FIGS. 2, 7, and 8, to attach the sealing cap 10 to the syringe 12, the connection portion 88 of the housing 48 of the sealing cap 10 is inserted into and threadedly engaged with a luer collar 122 surrounding a proximal end 28 of the distal tip 24. The distal tip 24 of the syringe 12 is received in the cavity 64 of the tip-engagement portion 54 of the sealing insert 46 with the inner surface of the sidewall 62 of the tip-engagement portion 54 of the sealing insert 46 creating a seal with at least a portion of the outer surface of the distal tip 24 and the stopper protrusion 100 of the tip-engagement portion 54 of the sealing insert 46 extending into and sealing the fluid passageway 26 of the distal tip 24.

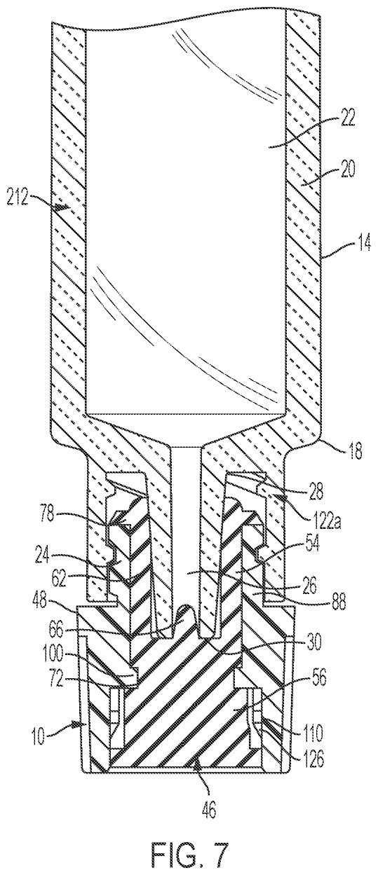

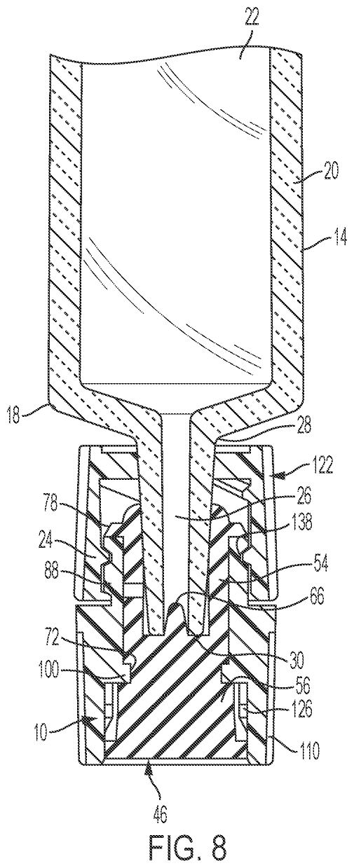

The luer collar 122 may be a separate component that is friction fitted onto the distal tip 24 of the syringe 12 (FIG. 8) or may be integrally molded with the distal tip 24 of the syringe 212 (FIG. 7). The luer collar 122 comprises a proximal end 132 attached to the distal face 32 of the syringe 12, an open distal end 134, and a sidewall 136 extending from the proximal end 132 to the distal end 134. The sidewall 136 of the luer collar 122 is spaced from and at least partially surrounds the distal tip 24 of the syringe 12 and an inner surface of the sidewall 136 of the luer collar 122 includes threads 138 that are engaged by the threads 120 of the connection portion 88 of the housing 48 of the sealing cap 10. The sidewall 136 of the luer collar 122 is concentric with the distal tip 24.

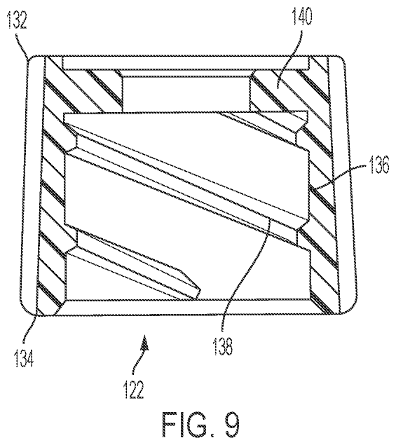

If the luer collar 122 is separate from the distal tip 24, a plurality of radially inward directed mounting projections 140 extend from the inner surface of the sidewall 136 of the luer collar 122 at the proximal end 132 of the luer collar 122 (FIG. 9). As the proximal end 132 of the luer collar 122 is urged in a proximal direction over the distal tip 24 of the syringe 12, the mounting projections 140 deflect slightly and frictionally engage the distal tip 24 to resist separation of luer collar 122 from the syringe 12.

Unintended separation of sealing cap 10 from the distal tip 24 is substantially prevented by the threaded engagement of sealing cap 10 with the luer collar 122. Further, the threaded engagement of sealing cap 10 with the luer collar 122 and the simultaneous engagement of the housing 48 with the sealing insert 46 positively prevent inadvertent breaking of the seal between the sealing insert 46 and the distal tip 24. However, the distal tip 24 of the syringe 12 can be readily accessed by rotating the sealing cap 10 to disengage the threads 120 of the sealing cap 10 from the threads 138 of the luer collar 122. Engagement of the optional anti-rotation ribs 124 of the housing-engagement portion 56 of the sealing insert 46 with the anti-rotation ribs 126 of the insert-engagement portion of the housing 48 ensures that the sealing insert 46 will rotate with the housing 48. Engagement of the groove 72 of the sealing insert 46 with the protrusion 100 of the housing 48 and the locking rib 78 of the sealing insert 46 with the proximal end 84 of the housing 48 ensures that the sealing insert 46 will move axially in response to the disengagement of the connection portion 88 of the housing 48 from the luer collar 122. Further, the locking rib 78 of the sealing insert 46 blocks and prevents the sealing insert from rolling and/or displacement, during assembly, during placement on the syringe 12 and/or removal from the syringe 12.

The sealing insert 46 may be made from any suitable material that is sufficiently flexible to compress and conform to the outer surface of the distal tip 24 of the syringe 12 to create a seal between the sealing insert 46 and the outer surface of the distal tip 24 of the syringe 12 that prevents air and contaminants from entering the fluid passageway 26 of the distal tip 24 of the syringe 12. For example, the sealing insert 46 may be made from polyethylene (PE), polypropylene (PP), or an elastomeric material, for example a rubber material such as styrene butadiene rubber, an isoprene bromobuytyl blend, or a thermoplastic elastomer. Suitable materials are inert to the medical solution to be filled into the medical container, have sufficient heat resistance to undergo the necessary sterilization procedures, and are sufficiently flexible to grip and release the distal tip 24 of the syringe 12. The sealing insert 46 may be manufactured by a molding process such as injection molding.

The material of the housing 48 has higher Young's modulus (stiffness), higher strength, higher hardness, and/or lower elasticity than the sealing insert 46. For example, the sealing insert 46 may be an elastomer having a Young's modulus of 0.0001 to 0.1 GPa, and the housing 48 may be a thermoplastic having a Young's modulus of 1-10 GPa.

The housing 48 may be made from any suitable material that provides sufficient compressive contact force on the sealing insert 46 to maintain a continuous seal between the sealing insert 46 and the outer surface of the distal tip 24 of the syringe 12 that prevents air and contaminants from entering the fluid passageway 26 of the distal tip 24 of the syringe 12. For example, the housing 48 may be made from a thermoplastic, such as, polycarbonate (PC), polyoxymethylene (POM), polybutylene terephthalate (PBT), polypropylene (PP), polyethylene (PE), polyvinylchloride (PVC), polystyrene, acrylonitrile butadiene styrene, styrene acrylonitrile, a fiber-reinforced polymer, a metal, or a combination thereof. The housing 48 may be made of polypropylene, as it is an easily moldable, strong and lightweight plastic material. Polypropylene further has the advantages of being highly impermeable to liquids and inert with respect to many chemicals, which prevents degradation of the housing 48 when it gets into contact with a medical solution. The housing 48 may be made of a transparent material which allows for verifying the correct assembly of the housing 48 and the sealing insert 46.

Whereas particular aspects of this invention have been described above for purposes of illustration, it will be evident to those skilled in the art that numerous variations of the details of the present invention may be made without departing from the invention.

1. A sealing cap for a syringe comprising a syringe barrel defining a reservoir for containing a medicament and a distal tip having a passageway in fluid communication with the reservoir, the sealing cap comprising:
a housing having an open proximal end, a distal end, and a sidewall extending from the proximal end to the distal end and defining a cavity; and
a sealing insert received within the cavity of the housing,
wherein the housing comprises a connection portion and an insert-engagement portion, and the sealing insert comprises a tip-engagement portion, and a housing-engagement portion,
the housing-engagement portion of the sealing insert is received within the insert-engagement portion of the housing, and the tip-engagement portion of the sealing insert is received within the connection portion of the housing,
an annular protrusion extends radially inwardly from the sidewall of the housing and is received in an annular groove positioned between and connecting the housing-engagement portion of the sealing insert and the tip-engagement portion of the sealing insert,
an annular locking rib extends around an outer periphery of a proximal end of the sealing insert and is positioned proximally beyond the proximal end of the housing, and
the engagement of the annular protrusion of the housing and the groove of the sealing insert and the engagement between the proximal end of the housing and the locking rib of the sealing insert hold the sealing insert within the housing.

2. The sealing cap of claim 1, wherein the tip-engagement portion comprises an open proximal end corresponding to the proximal end of the sealing insert, a closed distal end, and a sidewall defining a cavity extending between the proximal end of the tip-engagement portion and the distal end of the tip-engagement portion and adapted and dimensioned to receive the distal tip of the syringe, thereby forming a seal between an inner surface of the sidewall of the tip-engagement portion and an outer surface of the distal tip.

3. The sealing cap of claim 2, wherein a stopper protrusion extends from the distal end of the tip-engagement portion into the cavity of the tip-engagement portion, and is disposed and dimensioned to contact and at least partially extend into the passageway of the distal tip.

4. The sealing cap of claim 1, wherein the housing-engagement portion extends from the tip-engagement portion of the sealing insert in a distal direction and has a distal end that corresponds to the distal end of the sealing insert.

5. The sealing cap of claim 1, wherein the locking rib is spaced apart from the proximal end of the sealing insert such that a portion of the tip-engagement portion extends proximally beyond the locking rib.

6. The sealing cap of claim 1, wherein the locking rib has an outer peripheral face that is angled such that an outer diameter of a distal end of the locking rib is greater than an outer diameter of a proximal end of the locking rib.

7. The sealing cap of claim 1, wherein the locking rib has a trapezoidal cross-section.

8. The sealing cap of claim 1, wherein an outer diameter of the housing-engagement portion is greater than an outer diameter of the tip-engagement portion, and the locking rib has an outer diameter that is intermediate between the outer diameter of the tip-engagement portion and the outer diameter of the housing-engagement portion.

9. The sealing cap of claim 1, wherein the insert-engagement portion has an open proximal end, a distal end, and a sidewall defining a cavity adapted and dimensioned to receive the housing-engagement portion.

10. The sealing cap of claim 1, wherein the annular protrusion extends radially inwardly around the inner surface of the sidewall of the insert-engagement portion.

11. The sealing cap of claim 1, wherein the connection portion extends proximally from the insert-engagement portion and comprises an open proximal end, an open distal end, and a sidewall defining a cavity adapted to receive at least a portion of the tip-engagement portion, and threads extend around an outer peripheral surface of the sidewall of the connection portion.

12. The sealing cap of claim 1, wherein an outer diameter of the connection portion is less than an outer diameter of the insert-engagement portion.

13. The sealing cap of claim 1, wherein a diameter of the cavity of the housing that receives the tip-engagement portion is greater than the outer diameter of the tip-engagement portion, such that space is provided between the tip-engagement portion and the housing.

14. The sealing cap of claim 1, wherein the annular protrusion is spaced a distance from the distal end of the housing that is approximately equal to or greater than a distance between the groove and the distal end of the sealing insert.

15. The sealing cap of claim 1, wherein a diameter of the cavity of the housing at the annular protrusion is approximately equal to a diameter of the sealing insert within the groove.

16. The sealing cap of claim 1, wherein the locking rib is spaced a distance from the groove that is approximately equal to a distance between the annular protrusion and the proximal end of the housing.

17. The sealing cap of claim 1, further comprising a plurality of anti-rotation ribs extending axially in a longitudinal direction along an outer peripheral surface of the housing-engagement portion and/or a plurality of anti-rotation ribs extending axially in a longitudinal direction along an inner peripheral surface of the insert-engagement portion.

18. The sealing cap of claim 17, wherein the anti-rotation ribs of the housing-engagement portion are received in spaces between the anti-rotation ribs of the insert-engagement portion.

19. The sealing cap of claim 17, wherein a proximal end of the anti-rotation ribs of the housing-engagement portion and/or a distal end of the anti-rotation ribs of the housing-engagement portion are tapered or pointed.

20. The sealing cap of claim 1, wherein a material of the housing has a higher Young's modulus, higher strength, higher hardness, and/or lower elasticity than a material of the sealing insert.

21. The sealing cap of claim 1, wherein the sealing insert is made from a material selected from the group consisting of polyethylene (PE), polypropylene (PP), an elastomeric material, and the housing is made from a material selected from the group consisting of polycarbonate (PC), polyoxymethylene (POM), polybutylene terephthalate (PBT), polypropylene (PP), polyethylene (PE), polyvinylchloride (PVC), polystyrene, acrylonitrile butadiene styrene, styrene acrylonitrile, fiber-reinforced polymer, metal, and combinations thereof.

22.-25. (canceled)
